(19) United States
(12) Patent Application Publication
(10) Pub. No.: US 2010/0106046 A1
(43) Pub. Date: Apr. 29, 2010

(54) DEVICE AND METHOD FOR PREDICTING AND PREVENTING PULMONARY EDEMA AND MANAGEMENT OF TREATMENT THEREOF

(76) Inventors: Michael Shochat, Havazelet (IL); Pavel Rabinovich, Jerusalem (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(21) Appl. No.: 12/258,413

(22) Filed: Oct. 26, 2008

(51) Int. Cl. A61B 5/053

(52) U.S. Cl. 600/547

(57) ABSTRACT

A device and a method for preventive treatment of evolving pulmonary edema in patients which are at risk of complication associated with pulmonary edema which is based on the monitoring of internal thoracic impedance of the patient. The device extracts the internal thoracic impedance from measured trans-thoracic impedance and is relatively immune to variations in skin/electrode interface impedance. The method includes identification of a stage of interstitial edema development before the appearance of a clinical indication and the beginning of an appropriate medicinal treatment in accordance to variations of the monitored internal thoracic impedance. The method also indicates the appropriate moment for terminating the medicinal treatment and can be applied when the patient and his treating physician are positioned at remote locations.

### FIELD AND BACKGROUND OF THE INVENTION

The present invention relates to a method of medical treatment in general and to a method for prevention the clinical onset of the alveolar pulmonary edema in patients suffering diseases which are complicated with this pathological condition.

Pulmonary edema is an excessive accumulation of liquid in the lungs. It is a dangerous common complication of many, especially heart diseases (congestive heart failure, myocardial infarction, arterial hypertension, heart valves disease) and trauma, etc.

Results of treatment of evolving pulmonary edema can be improved significantly if it starts as early as possible to the appearance of clinical indications, at the stage of interstitial edema.

This is so because at the alveolar stage of pulmonary edema a vicious cycle has already started, it means that edematous liquid already fills lungs alveoli and forces them out of air. This impedes the blood O₂ saturation in the lungs and leads to general hypoxemia which may cause myocardial hypoxia with additional weakness of the cardiac muscle.

This in turn increases further the heart failure and the subsequent increase of the accumulation fluid into the alveolus which further decreases blood O₂ saturation and vice versa.

Early treatment intervention at the stage of interstitial edema before alveolar lung and vicious cycle starts have to prevent alveolar edema development and prevents its extremely painful and dangerous complications, thus for an efficient preventive treatment, pulmonary edema must be detected and treated in time, in its interstitial stage before the appearance of clinical signs.

The mentioned above objects of early detection of the interstitial stage of evolving pulmonary edema, the early onset of its preventive treatment and the treatment termination can be achieved provided that there exists a method for continuous monitoring the lungs condition of patients suffering from diseases which pose a risk of the pulmonary edema development.

Process of Pulmonary edema development is associated with fluid buildup in the lungs which causes therefore a decrease in electrical impedance across the lungs, a parameter which is termed herein after as lung impedance (LI).

FIG. 1 shows a correlation between LI and the amount of water in the lungs, accordingly, a persistent decrease in LI is indicative of a buildup of electrically conductive fluid in the lungs.

Thus, devices and method have been proposed to monitor the onset and advance of pulmonary edema by techniques of surface impedance plethysmography, in which the electrical impedance of the chest is measured by imposing an electrical current across the body via a set of attached electrodes and measuring the associated voltage difference which arises.

A main problem for early prediction of pulmonary edema development in prior art surface methods and devices was inability to diagnose small changes in LI at the stage of interstitial congestion (preclinical stage) because of low sensitivity of most existing medical devices and lack of appropriate methodology.

Typically, surface devices and methods in use today diagnose already existing alveolar edema and no method is available for prediction of the onset of pulmonary edema and for its preventive treatment.

Generally, the LI has to be extracted from a measured trans-thoracic impedance which includes parasitic contribution such as electrode/skin contact impedance and the impedance of skin, hence allegedly a more accurate and sensitive measurement of LI can be achieved by the use of implanted electrodes, which usually have been inserted in the body for other reasons e.g. for the purpose of defibrillation or heart pacing).

U.S. Pat. No. 6,595,927 to Pitts-Crick et al. describes a method and system for diagnosing and treating pulmonary congestion which is based on the deviation of a value of measured trans-thoracic impedance as by implanted electrodes from a background base-line.

U.S. Pat. No. 7,272,443 to Xiaoyi et al. describes the detection of overloads in liquid levels in the thorax and in ventricular myocardial mass of a patient as a result of the pulmonary edema, by impedance measurement across implanted electrodes,

U.S. Patent application No. 2002 01123674 to Picchi et al. describes a bioelectric impedance measurement by which is carried out by electrodes which are implanted in the lung tissue and which are supposed to overcome all drawbacks of prior LI measurements.

A description of using implanted electrodes to measure LI in order to follow the fluid status and thus the early monitoring pulmonary edema development is given in the publication; “Intrathoracic Impedance Monitoring in Patients With Heart Failure, Correlation With Fluid Status and Feasibility of Early Warning Preceding Hospitalization” by Chenk-Man Yu et al. (Circulation, Aug. 9, 2005; 112: 841-846).

Documentation which describes LI measurement with auxiliary electrode which are disposed across a chest of a patient includes Japanese Patent No. 200128748 to I. Tetsuya, which shows a lung water amount display device to detect the onset of pulmonary edema, and U.S. Pat. No. 7,096,061 to S. Arad which measure the trans thoracic impedance with auxiliary electrodes embedded in a chest belt.

Conclusions drawn from this collective prior art are: firstly, that while LI measurement with implanted electrode may provide the fluid status in the lung already in early stages of its accumulation, no such reliable and sensitive enough non invasive technique for LI measurement did exist.

Secondly, that regardless the LI measurement technique, no coherent method of preventive treatment for pulmonary edema exists which takes care of all the aspects of the episode and in particular the determination of the interstitial stage of evolving (onset) of pulmonary edema and the exact moment for impedance-guided starting a preventive treatment, the effect of medicine administered and determining of treatment termination.

The drawbacks associated with the use of implanted electrode for LI measurements (such as e.g. the implantation being an invasive procedure which is costly and causes inconvenience to the patient and which has low inherent sensitivity because electrode configuration inside the chest), have been recently removed when a device and a method for stable impedance plethysmography was described in U.S. Pat. No. 5,749,369 to P. Rabinovich et al., which is incorporated herein by reference for all purpose as if fully set forth herein.

The “369” patent, teaches a device in which at least two sets of electrodes are used, the first set of so called measuring electrodes, measures the thoracic impedance across two electrodes which are attached to opposite side of the patient chest, while the other set of electrodes so called references electrodes, measures the skin/electrode impedance which is included in the measured thoracic impedance across the first set of electrodes.

Subtracting the anterior and posterior skins electrode contact impedance from the impedance measured between the measuring electrodes yields corrected impedance whose change over time accurately reflects the change over time of the internal impedance of the biological object.

The present invention uses the “369” device to introduce a fundamental change in pulmonary edema treatment concept which takes care of all the aspects of the episode and in particular the determination of the interstitial stage of evolving of pulmonary edema and the exact moment for starting a preventive treatment, the effect of medicine administered and determining of treatment termination.

### SUMMARY

The present invention includes a device and method for prediction of alveolar stage of the pulmonary edema in absence of clinical signs in patients with diseases which render a risk of pulmonary edema development, for the preventive treatment and determination of individual doses of the administered medicines and for a knowledgeable decision of the treatment termination.

In accordance to the present invention there is provided a method for preventive treatment for pulmonary edema in a patient comprising the step of: (a) monitoring an internal thoracic impedance Z_{ITI} (which approximates LI) of the patient, (b) establishing an initial value of (Z_{ITI})ᵢ to said Z_{ITI}, (c) beginning a medical treatment against edema by administering the patient at least a first dose of medication when a value of said Z_{ITI} dropped beneath a threshold value while continuing monitoring and updating said Z_{ITI} and, (d) deciding on further treatment possibilities in accordance to said updated value of said Z_{ITI} wherein said possibilities are selected from the group consisting of administering an additional amount of said at least first dose of medicine, increasing the administered amount of medicine, administering a different medicine and termination of the treatment.

In accordance to the present invention there is provided a device for predicting of evolving of pulmonary edema in a patient which is based on measurements of internal thoracic impedance of the patient, the device comprising: (a) a first measuring electrode and a second measuring electrode to be attached to a patient outer skin, (b) a first set and a second set of reference electrodes to be attached to a patient outer skin, each set among said first set and second set of reference electrodes consist of a first electrode and a second electrode, (c) a first impedance measuring circuit for measuring a first impedance across said first and said second measurement electrodes, (d) a first impedance measuring assembly to evaluate a first contact impedance across contact of said first measuring electrode with said skin of said patient, (e) a second impedance measuring assembly to evaluate a second contact impedance across contact of said second measuring electrode with said skin of said patient, (f) a mechanism to calculate said internal thoracic impedance by subtracting a value which equals to the sum of said first contact impedance and said second contact impedance from said first impedance and, (g) a transmitter to transmit said values of said internal thoracic impedance.

In accordance to the present invention there is provided a system for preventing pulmonary edema comprising of: (a) a device for prediction of pulmonary edema in a patient which is based on measurements of internal thoracic impedance of the patient, (b) a transmitting unit to deliver data including values of said measured internal trans-thoracic impedance and, (c) a receiver to accept said delivered data.

It is an object of the present invention to introduce a novel and effective method of a preventive treatment of pulmonary edema.

It is another object of the present invention to introduce a method for early detection of an interstitial stage of an interstitial stage of evolving pulmonary edema prior to appearance of clinical indications.

It is yet another aim of the present invention to introduce a method to enable a knowledgeable decision on the termination of medical treatment of pulmonary edema. It is still another aim of the present invention to enable a patient to get a treatment to evolving pulmonary edema by a remote physician.

Other benefits of the present invention will become evident in conjunction with the accompanying figures.

### BRIEF DESCRIPTION OF THE FIGURES

FIG. 1 shows a correlation between water content in the lungs and the lung's electrical impedance (LI).

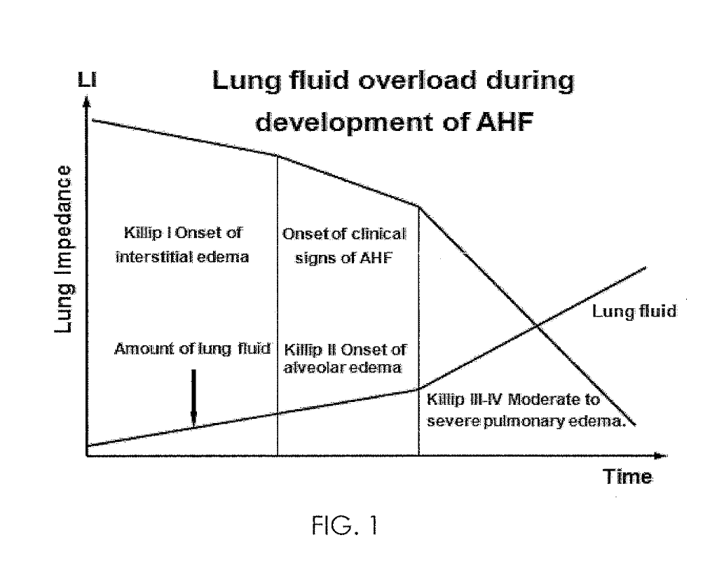

FIG. 2 shows a bock diagram of an edema guard monitor (EGM) in accordance to the present invention,

FIG. 3 shows a block diagram of an internal thoracic impedance measuring unit in EGM unit shown in FIG. 2,

FIG. 4 shows electrode configuration needed for internal thoracic impedance measurement by the unit shown in FIG. 3,

FIG. 5 shows a graphically, stages in pulmonary edema treatment in accordance to the present invention and,

FIG. 6 shows a flowchart of the stages of evolving pulmonary edema treatment in accordance with the present invention.

### DESCRIPTION OF PREFERRED EMBODIMENTS

The present embodiments herein are not intended to be exhaustive and to limit in any way the scope of the invention; rather they are used as examples for the clarification of the invention and for enabling of other skilled in the art to utilize its teaching.

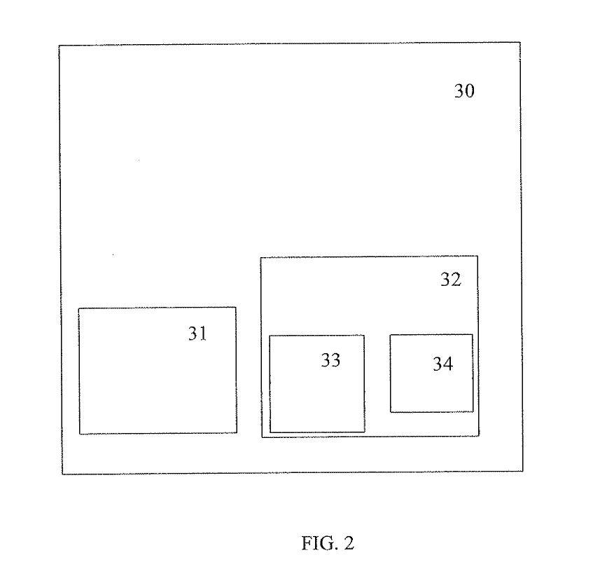

FIG. 2 depicts a block diagram of an edema guard monitor (EGM) device 30, suitable for the implementation of the method of the present invention.

In FIG. 2, device 30 includes an impedance measuring unit 31 and a telemetry unit 32 to transmit and receive data either via interface 33 which physically connects device 30 to an external processor or telephone line (not shown) or by a wireless transmitter 34 via a wireless link to a computer or a cellular device (not shown).

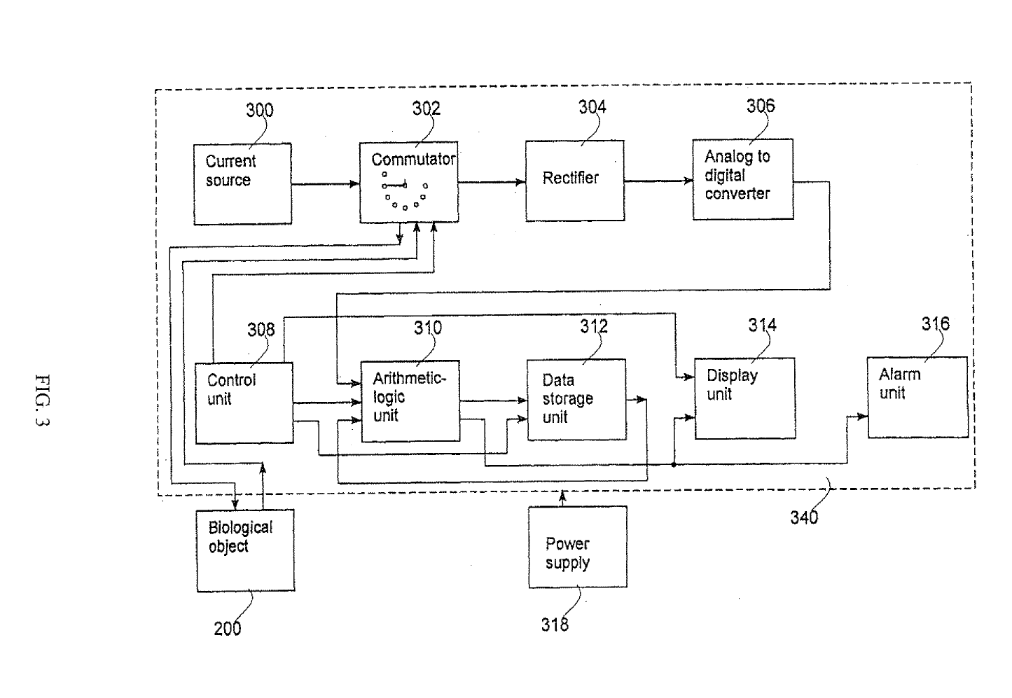

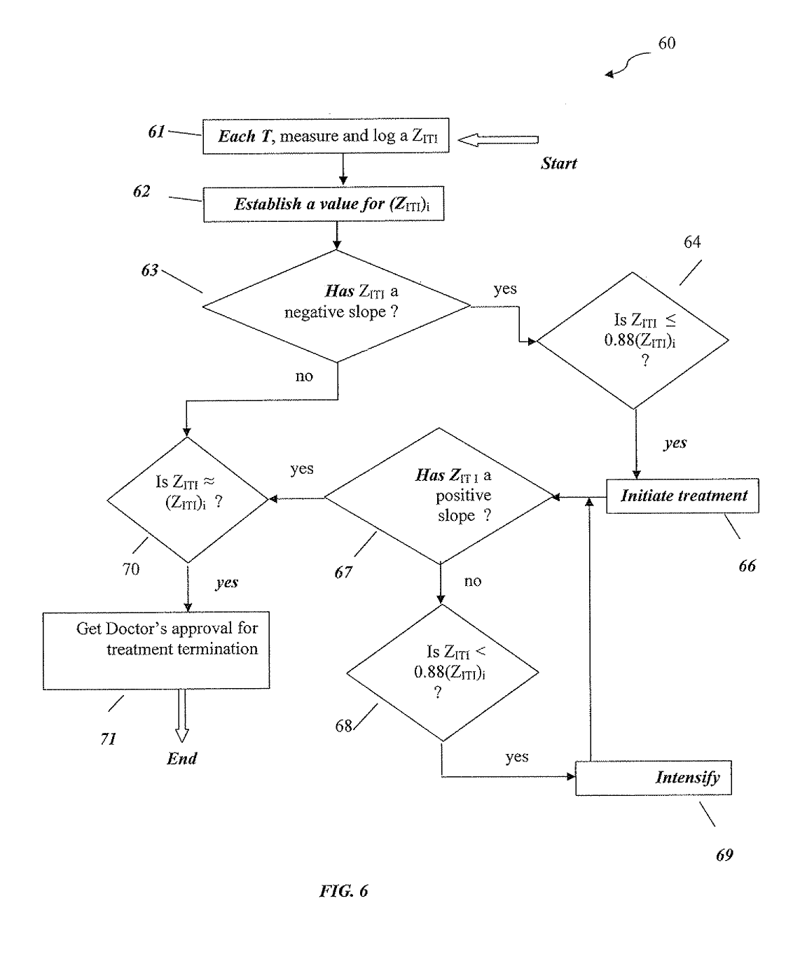

Impedance measuring unit 31 can be any prior art measuring device (outside the body or implanted) which has the appropriate measuring range, stability and resolution to extract LI from trans-thoracic measurement, preferably unit 31 consist of impedance measuring unit 340 shown in FIG. 6 in the “369” patent which is depicted in FIG. 3.

In FIG. 3, impedance measuring unit 31 includes: a current source 300; a commutator 302 for alternately connecting current source 300 to a distinct electrical circuit; a rectifier 304 for obtaining the absolute value of the signals representing the voltage drops across the various electrodes in a preferred configuration that will be detailed below; an analog to digital converter 306 for converting the signals to a digital form; an arithmetic-logic unit 310 for carrying out calculations; a data-storage unit 312 for storing data during the monitoring period; a display unit 314; an alarm unit 316; and a control unit 308 for controlling the operation of commutator 302, arithmetic-logic unit 310, data storage unit 312, and display unit 314. Arithmetic-logic unit 310 is electrically connected to data-storage unit 312, display unit 314, and alarm unit 316. Unit 31 is powered by means of a power supplier 318.

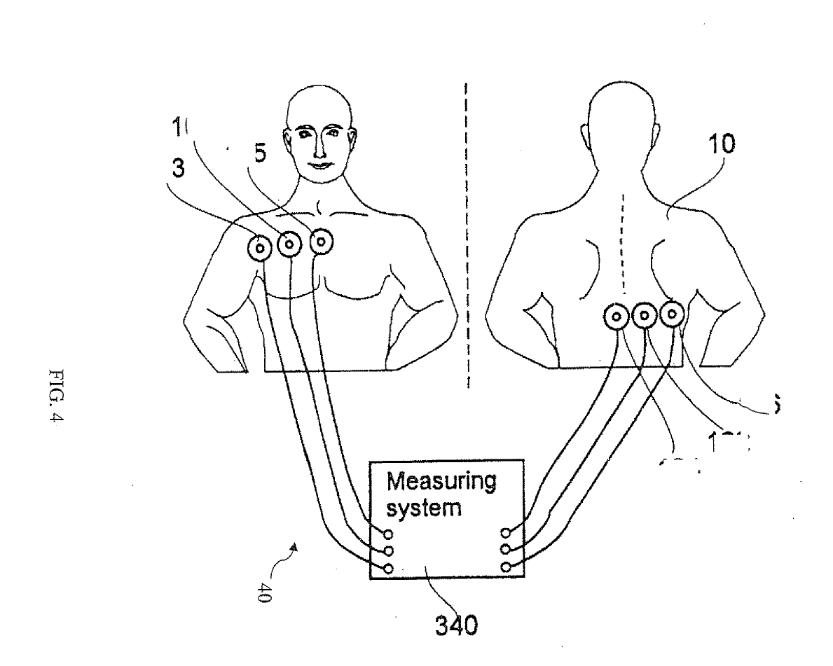

When using a device according to the present invention, electrical source 300 is alternately connected to each of the electrode pairs shown in FIG. 4 by means of commutator 302. The signal representing the voltage drop of a specific electrical circuit is fed into rectifier 304 which provides the absolute value of such voltage drop. An analog to digital converter converts the obtained signal to a digital form. The obtained signal is fed into arithmetic logic unit 310 and stored in data-storage unit 312. Then, control unit 308 orders commutator 302 to connect electrical source 300 to the next electrical circuit.

After storage unit 312 has received data from each of the seven electrical circuits, arithmetic logic unit 310 calculates an internal thoracic impedance ZIT, according to the method which will be described below.

Preferably, the process described above is carried out periodically, so that arithmetic logic unit 310 simultaneously calculates the values of the internal thoracic impedance Z_{ITI} as well as changes in Z_{ITI}. The change in Z_{ITI} may be calculated, for example, as the difference between the last value and the initial value or as a percentage from the initial value. The results of the calculations are transmitted to display unit 314, to data storage unit 312, and to alarm unit 316.

In the event that the value of Z_{ITI} has decreased below a critical value, and/or in the event that the change in Z_{ITI} has exceeded a critical value, alarm unit 316 is activated.

Data storage unit 312 may provide data for analysis during the monitoring period so as to monitor the progress of the disease.

Electrodes for trans thoracic impedance measurement and for Z_{ITI} evaluation, either implanted or surface electrodes are booked to unit 31 via suitable connector (not shown) and are attached to patient body 10 in any configuration known in the art, preferably, electrode configuration 40 shown in FIG. 4 and which is used in conjunction with measuring unit 31 is employed.

In configuration 40, measurement electrodes 1 and 2 are placed on opposite sides of the thorax of a patient 10. Electrode 1 is placed at the point of intersection of the right 3-intercostal space and the right mid clavicle line of patient 10. Measurement electrode 2 is placed on the back of patient 10, at the point of intersection of the right 8-intercostal space and the right scapular line.

Reference electrodes 3 and 5 are placed on opposite sides of electrode 1, and reference electrodes 4 and 6 are placed on opposite sides of electrode 2.

The internal thoracic impedance Z_{ITI} is automatically calculated by device 31 as described in the “369” patent from the trans thoracic impedance measured between electrodes 1 and 2; Z_{1,2}.

Z_{1,2} includes the skin and the electrode/skin contact impedances of both electrode 1 and electrode 2, hence in order to extract Z_{ITI} from measured Z_{1,2} these impedance contributions are subtracted from Z_{1,2} as follows:

Assume Z_{i,j} denotes the impedance measured between electrode i and electrode j as shown in FIG. 4, then

Z_{ITI}=Z_{1,2}−½(Z_{1,5}+Z_{1,3}−Z_{3,5})−½(Z_{2,4}+Z_{2,6}−Z_{4,6})

This is so because the term ½(Z_{1,5}+Z_{1,3}−Z_{3,5}) represents the skin and skin/electrode contact impedance of electrode 1 and the term ½(Z_{2,4}+Z_{2,6}−Z_{4,6}) represents the skin and skin/electrode contact impedance of electrode 2.

Z_{ITI} obtained according to the procedure described above was found to be a drift free value.

An important point to notice is that though Z_{ITI} may differ from the lung impedance (LI) because contribution of other inner tissues to the impedance, changes in Z_{ITI} of a patient posed at risk of pulmonary edema will nearly exclusively reflect the changes in LI because the impedance of the other inner tissues stay about constant.

It should be noted that other combinations of inter-electrode impedances which can be used to evaluate Z_{ITI} do not limit the present invention and are included within the scope thereof.

When a prediction of a patient at home, in hospital or in a health care center suggests a suspicion for an illness which may induce development of pulmonary edema when clinical signs for pulmonary edema are yet absent, the patient is attached to EGM 30 and monitoring of Z_{ITI} begins.

It should be noted however that the method described below is not limited to EGM 30, rather it can be implemented with other suitable device for impedance plethysmography and by using different electrodes (regardless whether external or implanted) which measure Z_{ITI} or its equivalent

Measurements of Z_{ITI} should be taken repetitively, e.g. at least each 30 minutes. The average value of initially measured Z_{ITI} is designated as (Z_{ITI})ᵢ and serves as a reference point to which further measured Z_{ITI} are compared.

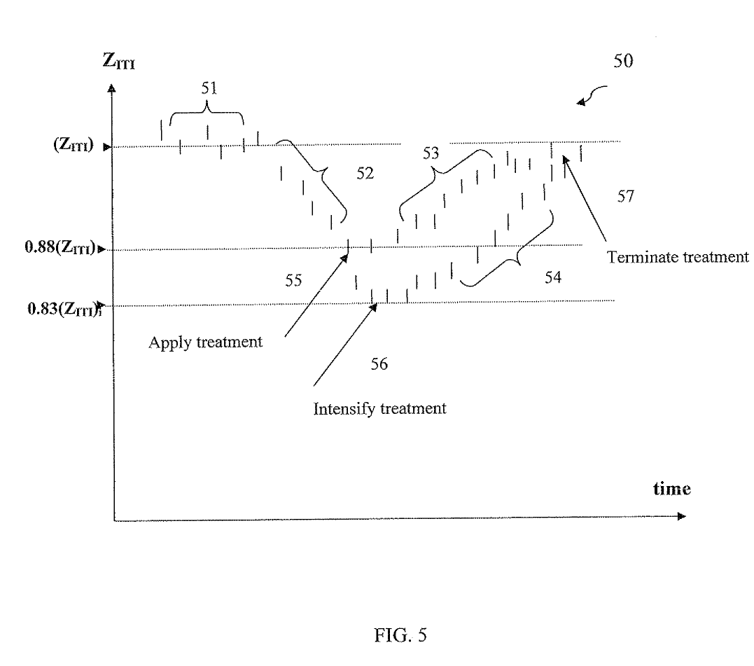

Applicants of the present invention correlated quantitatively the value of Z_{ITI} to stages in the evolution of pulmonary edema, their finding are shown in graph 50 of FIG. 5 where the changes of Z_{ITI} of a patient which develops pulmonary edema with time are shown.

Stages of early prediction, subsequent treatment and terminating the treatment of evolving pulmonary edema which are based on continuously or periodically monitoring Z_{ITI} as described above, are shown in FIG. 5 as well.

In graph 50, Z_{ITI} data is shown by error bars. The flat segment 51 in the value of Z_{ITI} reflects the stage prior to interstitial edema from which a reference base line and the value of (Z_{ITI})ᵢ is deduced, the beginning of the stage of interstitial edema is indicated by a decrease in Z_{ITI}.

The intensifying of interstitial edema which is shown in section 52 has no clinical signs but is detectable by the monotonically drop in Z_{ITI}.

Z_{ITI} values which correspond to a relative decrease of less than 12% then the value of (Z_{ITI})ᵢ reflect interstitial pre-clinical (as revealed by X-rays investigation) stages of evolving pulmonary edema which are not dangerous to the patient while a decrease of beyond about 12% from the value of (Z_{ITI})ᵢ indicates a stage of transformation from interstitial edema to alveolar edema (which is also observed by X-rays investigation), this stage has also no clinical signs.

When alveolar lung edema initiates, Z_{ITI} decreases by at least about 17% then the initial (Z_{ITI})ᵢ.

Applicants of the present invention disclose that the most effective preventive treatment for evolving pulmonary edema is accomplished if medical treatment starts at the preclinical stage, preferably at the stage of interstitial edema, hence a threshold of about 12% in the relative decrease of Z_{ITI} with respect to (Z_{ITI})ᵢ as indicated by the line designating 0.88(Z_{ITI})ᵢ shown in FIG. 5, is selected for the instant of the beginning of medical treatment for evolving pulmonary edema.

It should be noted however, that the exact value of the threshold does not limit the present invention and different values for said threshold can be used as a function of the state of health of the patient such as e.g. Z_{ITI} values of between about 10% to about 15% lower then the value of (Z_{ITI})ᵢ.

If the threshold value of Z_{ITI} has been reached a treatment which is based on the administration of the proper medication begins. This moment is indicated by arrow 55 in FIG. 5, if within a next preselected period of time, e.g. within half an hour since said administration of the medication the value of Z_{ITI} further decreases in spite the treatment, a more aggressive dose of medication, or another medication are administered as shown by arrow 56 in FIG. 5.

If on the other hand, as a result of the administration of the medications, variations in the value of Z_{ITI} reverse direction and the value of Z_{ITI} increases as shown in sections 53 and 54 in FIG. 5, said administration should be continued until the value Z_{ITI} of closely meets the value of (Z_{ITI})ᵢ, Then the treatment is terminated as shown in arrow 57 in FIG. 5.

The method which was detailed above is summarized in flowchart 60 shown in FIG. 6.

In flowchart 60, the method starts in measuring Z_{ITI} repeatedly each time interval T and logging the measured values as shown in stage 61. The method continues to stage 62 where a value of (Z_{ITI})ᵢ is established from the initial measurements which were taken.

In stage 63 which proceeds, it is inspected whether Z_{ITI} decreases, if Z_{ITI} does not decrease for a sufficient time, the Doctor may decide on treatment termination (or prohibiting treatment initiation in this case) as shown in stage 70, otherwise it is probed whether Z_{ITI} reached the threshold value of 0.88(Z_{ITI})ᵢ needed for treatment initiation as shown in stage 64.

If Z_{ITI} reached or passed said threshold, treatment begins as shown in stage 66, if it did not, treatment will not yet be started.

Stage 67 which proceed, probes whether Z_{ITI} increases as a result of the applied treatment. If it increased it will be checked whether the value of Z_{ITI} nearly reached again that of (Z_{ITI})ᵢ and if so, treatment ends (subjected to the doctor approval) as shown in stage 70

If however Z_{ITI} does not increase, stage 68 probes its value in order to decide on a more aggressive treatment designated as stage 69, which should be executed in case Z_{ITI} reached or passed a second threshold value of 0.83(Z_{ITI})ᵢ.

When an improvement is observed as a result of the aggressive treatment and the value of Z_{ITI} is no more smaller then about 0.88(Z_{ITI})ᵢ, the treatment can be alleviated to the level which corresponds to stage 66.

The treatment ends, in subjection to the doctor approval when the value of Z_{ITI} returns to the value of (Z_{ITI})ᵢ as shown in stage 70.

A preferred implementation of the present invention is occurs when the patient and the doctor who predicts the patient's evolving pulmonary edema are located in remote places, e.g. at different department of a hospital, at two different hospitals, or when the patient resides at home while the doctor which is positioned remotely orders on its emergent hospitalized in accordance to the value of the Z_{ITI} which he receives from telemetry unit 32 of device 30.

Such information can be transferred via any known wired or wireless mechanism of data-link such as modem, cellular, internet or RF communication.

### EXAMPLE

The utility of the present invention is demonstrated by the following example:

68 patients with acute myocardial infarction were admitted to 3 cardiology departments in Israel. All 68 patients suffered from acute myocardial infarction (AMI). The inclusion criterion to the test were absents of clinical and Roentgenological signs of lung edema at the beginning of monitoring and the decrease of the value of Z_{ITI} value by 12% (and more) compared to the value of (Z_{ITI})ᵢ during the monitoring. According X-ray examinations all patients were at interstitial stage of evolving pulmonary edema.

The 68 patients were then divided into 2 groups of 34 patients each. The first group of 34 patients was treated immediately after the value of Z_{ITI} decreased beyond the said 12% threshold (Preventive Treatment Group-PT group). The remaining 34 patients were treated conventionally, i.e. only after the appearance of Pulmonary Edema clinical signs (No-preventative Treatment Group—NPT group). Such parameters as mean age, the male/female ratio, height, weight and initial Z_{ITI} were identical in two groups.

The pulmonary edema did not develop in 29 of 34 patients who received the preventive treatment (PT group) and developed in mild form in the remaining 5 patients of this group. No fatal outcomes were observed. X-ray examinations have confirmed absence alveolar edema in 29 patients without clinical signs of edema and development alveolar edema in 5 rest patients.

The pulmonary edema was developed in all 34 patients who have not received the preventive treatment (NPT group) with 4 fatal cases (p<0.001). In all patients were found signs of alveolar edema on X-ray examinations.

While the invention has been described with respect to a limited number of embodiments, it will be appreciated that many variations, modifications and other applications of the invention may be made without departing from the scope and spirit of the invention.

What is claimed is:

1. A method for preventive treatment for evolving pulmonary edema in a patient comprising the step of:
(a) monitoring an electrical lung impedance of the patient,
(b) establishing an initial value to said lung impedance,
(c) beginning a medical treatment against evolving edema by administering the patient at least a first dose of medication when a value of said 1 lung impedance dropped beneath a threshold value while continuing monitoring and updating said lung impedance and,
(d) deciding on further treatment possibilities in accordance to said updated value of said lung impedance wherein said possibilities are selected from the group consisting of administering an additional amount of said at least first dose of medicine, increasing the administered amount of medicine, administering a different medicine and termination of the treatment.

2. The method as in claim 1 wherein said lung impedance is a fraction of a measured internal thoracic impedance (Z_{ITI}) of the patient.

3. The method as in claim 1 wherein said patient suffers a disease which poses a risk of pulmonary edema development.

4. The method as in claim 2 wherein period of time between consecutive Z_{ITI} measurements in said monitoring is no more then about 30 minutes.

5. The method as in claim 2 wherein said initial value of Z_{ITI} is a value of the patient's internal thoracic impedance before onset of interstitial lung edema ((Z_{ITI})ᵢ)

6. The method as in claim 5 wherein said threshold value is a value that is between about 10% to about 15% lower then said initial value of Z_{ITI}.

7. The method as in claim 2 wherein said beginning of preventive medical treatment takes place in spite of an absence of clinical signs for edema in the patient.

8. The method as in claim 2 wherein data which includes said monitored values of Z_{ITI} are transmitted to a doctor which is positioned remotely from the patient.

9. The method as in claim 8 wherein said transmitting of data is accomplished by techniques which are selected from the group consisting of a wired and wireless communication.

10. The method as in claim 5 wherein said termination of treatment takes place when updated value of said Z_{ITI} is about equal to said value of (Z_{ITI})ᵢ.

11. The method as in claim 2 wherein at least one value of Z_{ITI} of the patient is measured at a location which is selected from the group consisting of a hospital, a health care center and patient's residence.

12. A device for predicting of evolving of pulmonary edema in a patient which is based on measurements of internal thoracic impedance of the patient, the device comprising:
(a) a first measuring electrode and a second measuring electrode to be attached to a patient outer skin,
(b) a first set and a second set of reference electrodes to be attached to a patient outer skin, each set among said first set and second set of reference electrodes consist of a first electrode and a second electrode,
(c) a first impedance measuring circuit for measuring a first impedance across said first and said second measurement electrodes,
(d) a first impedance measuring assembly to evaluate a first contact impedance across contact of said first measuring electrode with said skin of said patient,
(e) a second impedance measuring assembly to evaluate a second contact impedance across contact of said second measuring electrode with said skin of said patient,
(f) a mechanism to calculate said internal thoracic impedance by subtracting a value which equals to the sum of said first contact impedance and said second contact impedance from said first impedance and,
(g) a transmitter to transmit said values of said internal thoracic impedance.

13. The device as in claim 12 further comprising:
(h) an interface card for interfacing the device to a compute and,
(i) an alarm unit to indicate when value of said internal thoracic impedance drops below a preselected threshold.

14. The device as in claim 13 wherein said preselected threshold is a value of the patient's internal thoracic impedance before onset of interstitial lung edema.

15. The device as in claim 12 wherein said transmitter delivers data via a communication technique which is selected from the group consisting of wired communication and wireless communication.

16. A system for prevention of pulmonary edema comprising of:
(a) a device for predicting of interstitial stage of evolving pulmonary edema in a patient which is based on measurements of internal thoracic impedance of the patient,
(b) a transmitting unit to deliver data including values of said measured internal thoracic impedance and,
(c) a receiver to accept said delivered data.

17. The system as in claim 16 wherein said transmitting unit includes a computer.

18. The system as in claim 16 wherein said receiver includes a computer.
